(19) United States
(12) Patent Application Publication
(10) Pub. No.: US 2025/0391536 A1
(43) Pub. Date: Dec. 25, 2025

(54) REMOTE APPROVAL, DISPENSING, AND REVIEW OF OPIOID TREATMENT DOSES

(71) Applicant: Opio Connect, Inc., Kalispell, MT (US)

(72) Inventors: Amber Norbeck, Kalispell, MT (US); Michael Pokorny, Kalispell, MT (US); Kelly Chu, Kalispell, MT (US); Thomas Hoffmann, Kalispell, MT (US)

(21) Appl. No.: 19/306,568

(22) Filed: Aug. 21, 2025

Related U.S. Application Data
(63) Continuation-in-part of application No. 19/208,950, filed on May 15, 2025.
(63) Continuation-in-part of application No. 19/211,700, filed on May 19, 2025.
(60) Provisional application No. 63/647,719, filed on May 15, 2024.
(60) Provisional application No. 63/648,895, filed on May 17, 2024.

(51) Int. Cl. G16H 20/13; A61J 7/00

(52) U.S. Cl. CPC G16H 20/13; A61J 7/0084; A61J 2200/70

(57) ABSTRACT

A method for remotely approving and dispensing medications directly to a patient for opioid addiction treatment. The method includes determining an identity of the patient, by a biometric sensing component and establishing remote communication between the patient and a medical official certified for treating opioid addiction. The method also includes transmitting a medical record corresponding to the patient to the medical official, generating, by the medical official, the opioid addiction treatment, generating an event record made up of the identity of the patient, the opioid addiction treatment, the medical record, and the medical official, and transmitting the event record to a dosing location.

### CROSS-REFERENCE TO RELATED APPLICATIONS

This application is a continuation-in-part and claims benefit of U.S. patent application Ser. No. 19/208,950 filed May 15, 2025, which is a non-provisional and claims benefit of U.S. Provisional Application No. 63/647,719 filed May 15, 2024, the specification(s) of which are incorporated herein in their entirety by reference.

This application is also a continuation-in-part and claims benefit of U.S. patent application Ser. No. 19/211,700 filed May 19, 2025, which is a non-provisional and claims benefit of U.S. Provisional Application No. 63/648,895 filed May 17, 2024, the specification(s) of which are incorporated herein in their entirety by reference.

### FIELD OF THE INVENTION

The present invention is directed to a process for providing opioid treatment medication to underserved and/or remote areas.

### BACKGROUND OF THE INVENTION

For many underserved communities, it is difficult to provide proper opioid treatment for local citizens suffering from addiction. This is because only officials who are authorized can diagnose and prescribe opioid treatment drugs (e.g. methadone) to patients. For areas of lower population, this requires some patients to travel long distances to see an authorized official physically. Telemedicine services exist but do not have the proper certifications to allow for the prescription of opioid treatment drugs like methadone. Thus, there exists a present need for providing opioid treatment medication to underserved and/or remote areas.

### BRIEF SUMMARY OF THE INVENTION

It is an objective of the present invention to provide methods and systems that allow for providing opioid treatment medication to underserved and/or remote areas, as specified in the independent claims. Embodiments of the invention are given in the dependent claims. Embodiments of the present invention can be freely combined with each other if they are not mutually exclusive.

The present invention features a method for remotely approving opioid addiction treatment to a patient. The method may comprise determining an identity of the patient, by a biometric sensing component. The method may further comprise establishing remote communication between the patient and a medical official. The medical official may be certified for treating opioid addiction. The method may further comprise transmitting a medical record corresponding to the patient to the medical official. The method may further comprise generating, by the medical official, the opioid addiction treatment. The method may further comprise generating an event record comprising the identity of the patient, the opioid addiction treatment, the medical record, and the medical official. The method may further comprise transmitting the event record to a dosing location.

The present invention features a computing system for remotely approving and dispensing treatment directly to a patient with opioid addiction. The system may comprise a communication component configured to communicatively couple to a plurality of patient devices, a processor, and a memory component comprising computer-readable instructions. The computer-readable instructions may comprise accepting biometric data from a patient device. The computer-readable instructions may further comprise confirming an identity of the patient based on the biometric data. The computer-readable instructions may further comprise accessing a medical record of the patient based on the confirmed identity. The computer-readable instructions may further comprise establishing, by the communication component, communication between a medical official and the patient through the patient device. The medical official may be certified for treating opioid addiction. The computer-readable instructions may further comprise accepting the opioid addiction treatment from the medical official. The computer-readable instructions may further comprise generating an event record comprising the identity of the patient, the opioid addiction treatment, the medical record, and the medical official. The computer-readable instructions may further comprise transmitting the event record to a dosing location.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a flow chart diagram of the process for remotely approving and dispensing medications directly to a patient for opioid addiction treatment of the present invention.

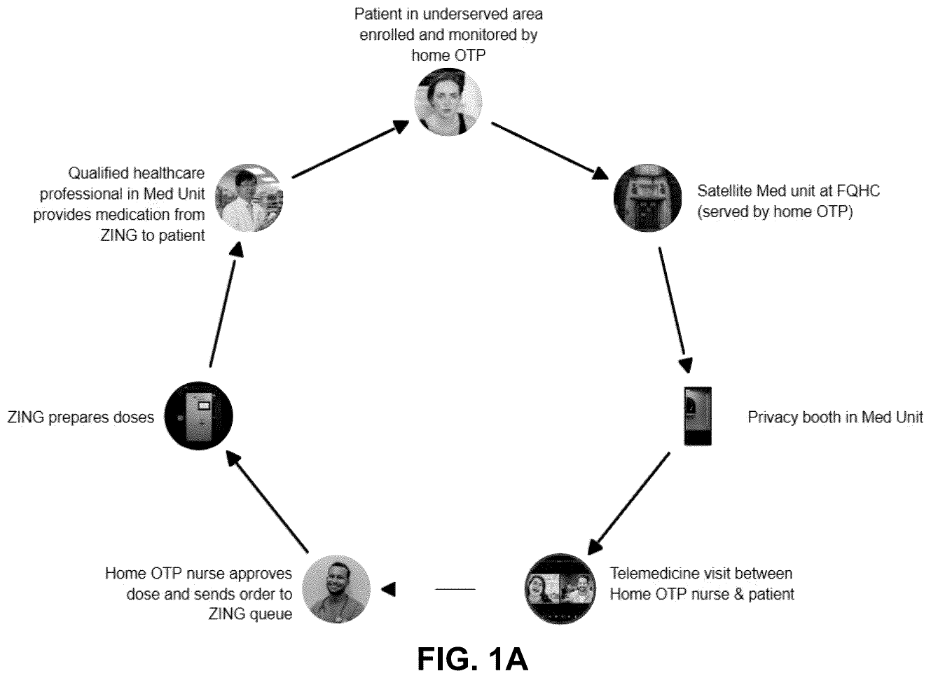

FIG. 1B shows an alternate flow chart diagram of the process for remotely approving and dispensing medications directly to a patient for opioid addiction treatment of the present invention.

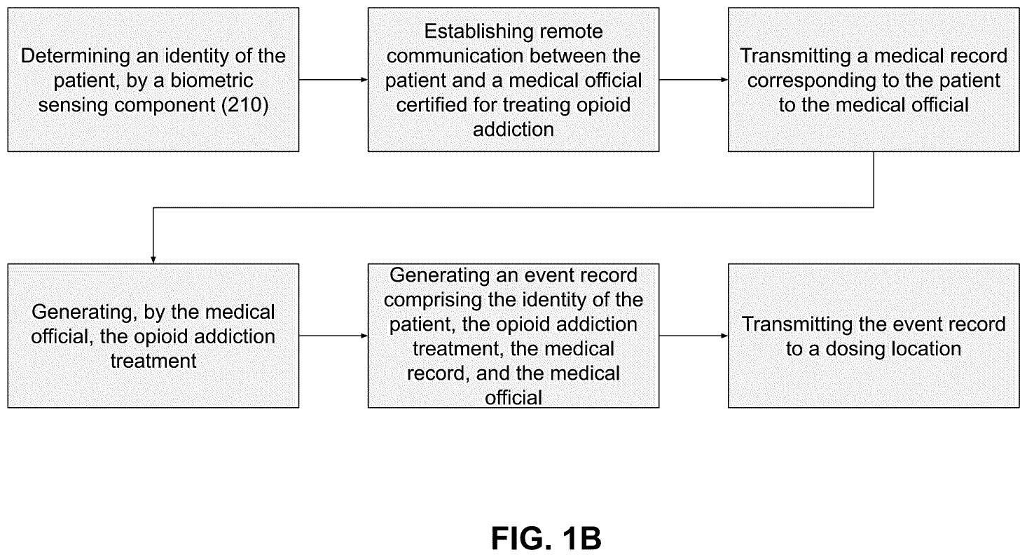

FIG. 2 shows a schematic diagram of a system for remotely approving and dispensing medications directly to a patient for opioid addiction treatment of the present invention.

FIG. 3A shows a photograph of the user interface implemented in the system for remotely approving and dispensing medications directly to a patient for opioid addiction treatment of the present invention.

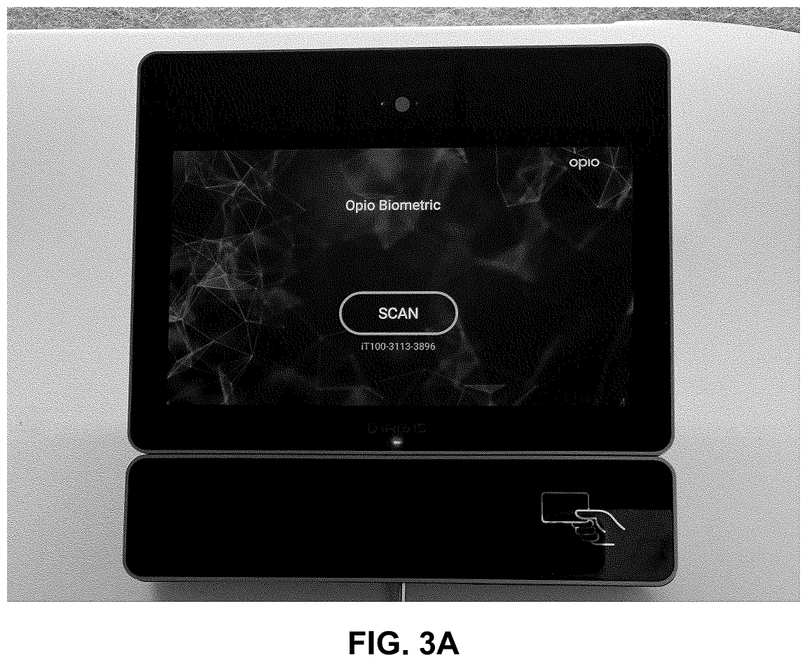

FIG. 3B shows a photograph of the user interface and screen implemented in the system for remotely approving and dispensing medications directly to a patient for opioid addiction treatment of the present invention.

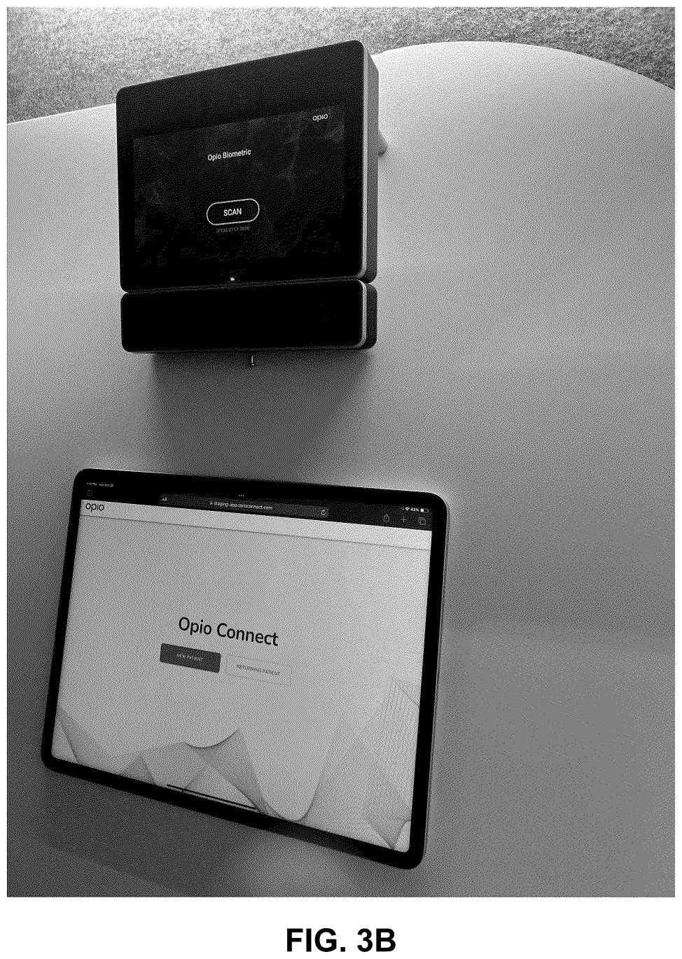

### DETAILED DESCRIPTION OF THE INVENTION

Following is a list of elements corresponding to a particular element referred to herein:
100 system
110 hub communication component
120 hub processor
130 hub memory component
200 patient device
210 biometric sensing component
220 patient device communication component
230 patient device processor
240 patient device memory component
1000 system

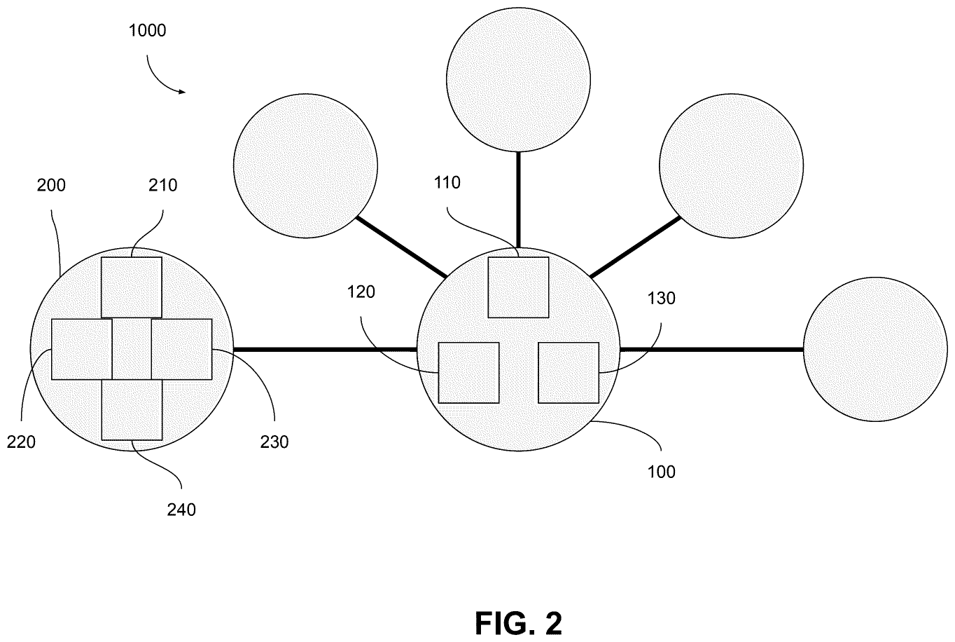

Referring now to FIG. 2B, the present invention features a method for remotely approving and dispensing medications directly to a patient for opioid addiction treatment to a patient. In some embodiments, the method may comprise determining an identity of the patient, by a biometric sensing component (210). The method may further comprise establishing remote communication between the patient and a medical official. The medical official may be certified for treating opioid addiction. The method may further comprise transmitting a medical record corresponding to the patient to the medical official. The method may further comprise generating, by the medical official, the opioid addiction treatment. The method may further comprise generating an event record comprising the identity of the patient, the opioid addiction treatment, the medical record, and the medical official. The method may further comprise transmitting the event record to a dosing location.

In some embodiments, the biometric sensing component (210) may comprise a fingerprint sensor, a retina scanner, a facial recognition component, or a combination thereof. In some embodiments, the method may further comprise providing a private booth configured to provide the remote communication between the patient and the medical official. In some embodiments, the opioid addiction treatment may comprise a dose of methadone, a dose of buprenorphine, or a combination thereof. In some embodiments, the medical official may comprise a pharmacist, a nurse, or a doctor. In some embodiments, the dosing location may comprise an automated dosage dispensing device.

The present invention features a computing system (100) for remotely approving and dispensing medications directly to a patient for opioid addiction treatment to a patient. In some embodiments, the system (100) may comprise a communication component (110) configured to communicatively couple to a plurality of patient devices. The system (100) may further comprise a processor (120) operatively coupled to the communication component (110), configured to execute computer-readable instructions. The system (100) may further comprise a memory component (130) operatively coupled to the processor (120), comprising computer-readable instructions.

The computer-readable instructions may comprise accepting biometric data from a patient device (200) of the plurality of patient devices. The computer-readable instructions may further comprise confirming an identity of the patient based on the biometric data. The computer-readable instructions may further comprise accessing a medical record of the patient based on the confirmed identity. The computer-readable instructions may further comprise establishing, by the communication component (110), communication between a medical official and the patient through the patient device (200). The medical official may be certified for treating opioid addiction. The computer-readable instructions may further comprise accepting the opioid addiction treatment from the medical official. The computer-readable instructions may further comprise generating an event record comprising the identity of the patient, the opioid addiction treatment, the medical record, and the medical official. The computer-readable instructions may further comprise transmitting the event record to a dosing location.

In some embodiments, the biometric data may comprise fingerprint data, retina data, facial data, or a combination thereof. In some embodiments, the plurality of patient devices may comprise personal computing devices, portable computing devices, private booths, or a combination thereof. In some embodiments, the opioid addiction treatment may comprise a dose of methadone, a dose of buprenorphine, or a combination thereof. In some embodiments, the medical official may comprise a pharmacist, a nurse, or a doctor. In some embodiments, the dosing location may comprise an automated dosage dispensing device.

Referring now to FIG. 2, the present invention features a computing system (1000) for remotely approving and dispensing medications directly to a patient for opioid addiction treatment to a patient. In some embodiments, the system (1000) may comprise a plurality of patient devices. Each patient device (200) may comprise a biometric sensing component (210) configured to accept biometric data from the patient. Each patient device (200) may further comprise a first communication component (220). Each patient device (200) may further comprise a first processor (230) operatively coupled to the biometric sensing component (210) and the first communication component (220), configured to execute computer-readable instructions. Each patient device (200) may further comprise a first memory component (240) operatively coupled to the first processor (230), comprising computer-readable instructions. The computer-readable instructions of the first memory component (240) may comprise accepting the biometric data from the biometric sensing component (210). The computer-readable instructions of the first memory component (240) may further comprise transmitting, by the first communication component (220), the biometric data to a hub computing system (100).

The system (1000) may further comprise the hub computing system (100). The hub computing system (100) may comprise a second communication (110) component configured to communicatively couple to the plurality of patient devices. The hub computing system (100) may further comprise a second processor (120) operatively coupled to the second communication component (110), configured to execute computer-readable instructions. The hub computing system (100) may further comprise a second memory component (130) operatively coupled to the second processor (120), comprising computer-readable instructions.

The computer-readable instructions of the second memory component (130) may comprise accepting biometric data from a patient device (200) of the plurality of patient devices. The computer-readable instructions of the second memory component (130) may further comprise confirming an identity of the patient based on the biometric data. The computer-readable instructions of the second memory component (130) may further comprise accessing a medical record of the patient based on the confirmed identity. The computer-readable instructions of the second memory component (130) may further comprise establishing, by the second communication component (110), communication between a medical official and the patient through the first communication component (220) of the patient device (200). The medical official may be certified for treating opioid addiction. The computer-readable instructions of the second memory component (130) may further comprise accepting the opioid addiction treatment from the medical official. The computer-readable instructions of the second memory component (130) may further comprise generating an event record comprising the identity of the patient, the opioid addiction treatment, the medical record, and the medical official. The computer-readable instructions of the second memory component (130) may further comprise transmitting the event record to a dosing location.

In some embodiments, the biometric sensing component (210) comprises a fingerprint sensor, a retina scanner, a facial recognition component, or a combination thereof. In some embodiments, the plurality of patient devices may comprise personal computing devices, portable computing devices, private booths, or a combination thereof. In some embodiments, the opioid addiction treatment may comprise a dose of methadone, a dose of buprenorphine, or a combination thereof. In some embodiments, the medical official may comprise a pharmacist, a nurse, or a doctor. In some embodiments, the dosing location may comprise an automated dosage dispensing device.

### EXAMPLE

The following is a non-limiting example of the present invention. It is to be understood that said example is not intended to limit the present invention in any way. Equivalents or substitutes are within the scope of the present invention.

The Hub and Spoke procedure is executed by a remote dosing nurse serving patients at 3 to 5 sites throughout the day. Patients from multiple locations are organized into a centralized queue. Only patients who require attention or are in an active session are displayed (not past sessions) in a retrievable list. Each patient has a display ID that is displayed alongside their name and location. The centralized queue supports multiple concurrent video sessions across multiple sites for multitasking; the nurse can continue to see any active video session with a patient, even if on with another. The nurse can view all active patient sessions on one screen, exiting and returning to sessions as needed. Upon session completion, the nurse must click a button to formally end the session (everything is time-stamped).

In an interaction between a nurse and a patient, the patient's name, date of birth, and consent (if the patient is new) are gathered. Additionally, a biometric patient identity verification is generated for new patients based on a facial ID. For returning patients, their ID is confirmed with a facial scan. Based on this information, the nurse pulls up the patient's profile, including the patient's response to clinic questions. The nurse sends an order for opioid addiction medicine to a dosing location tied to the patient's location.

The “batch all” configuration in Methasoft® is utilized, processing the first ordered doe first. The first observed dose is processed immediately and requires approval from the nurse before the single dose is released to the patient. Take-out doses automatically enter a paused state. For grouped doses (e.g., take-home doses), the nurse views a grouped photo of doses and, after a visual inspection, the nurse clicks a single “Approve and Dispense Observed Dose” button, displaying the patient ID for confirmation and unlocking the door (ASAP) for the observed dose. The doses are not pushed from the buffer into the tray drawer UNTIL the nurse hits the button.

The nurse observes that the patient takes the doses and stores them securely. The nurse records that the doses are successfully retrieved. If so, the nurse clicks a button to confirm this. If not, the nurse signals that the doses need to be quarantined. After observing the patient taking their dose, the nurse clicks “Resume” to process take-out doses. While automation processes doses, the nurse joins other video sessions and continues monitoring. All actions are timestamped. The nurse maintains full visibility of patient medication ingestion.

On the patient side, new patients submit their information and establish their biometric identification and unique ID for future identity confirmation. Returning patients use this information to confirm their ID in the system. The patient answers clinic-required questions while waiting for the nurse. The patient interacts with the nurse via video conference. The patient confirms, receives, and ingests the first observed dose, then disposes of the empty bottle into a secure garbage container. Remaining doses are removed and secured in a lockbox. All interactions are timestamped.

In a first example, a returning patient enters a booth implementing the process of the presently claimed invention. The booth requests the patient's first name, last name, and date of birth. The booth then requests the patient's identification number (ID) to confirm the identity of the patient in the system's records. If the patient does not have their ID, the booth takes a picture of the patient and confirms their identity in the system's records with a facial data analysis. The session with the nurse begins when the patient's identity is confirmed and the nurse joins the session.

During the session, the patient's name, ID, and location are on-screen at all times during the session. If necessary, the photograph is shown with its authentication score to determine if the patient's identity was properly verified. If necessary, the patient's information is updated by the nurse from the patient's profile. If necessary, the current patient profile is changed from an incorrect profile to the correct one. If the patient's photograph is too blurry, the nurse retakes the photograph through the booth. The nurse verifies the patient and proceeds with the session.

In the case of the nurse changing the patient's profile to a different profile, the nurse selects “Select New Patient.” The patient list appears on the screen. The nurse scrolls and selects the patient profile with information matching the information of the patient. Upon selecting a new patient profile, the session details are traced to the new patient profile and the nurse re-verifies the patient's identity. The patient's photograph is now saved to the new patient profile. If necessary, the booth takes additional new photographs of the patient.

In a second example, a new patient enters a booth implementing the process of the presently claimed invention. The booth requests the patient's first name, last name, and date of birth. The booth prompts the patient to agree to the terms and conditions to use the system. The booth requests and takes an image of the patient's ID card, if they have one. If they do not have an ID card, this step is skipped. The session with the nurse begins when the patient's onboarding process is complete and the nurse joins the session. The photograph of the ID card, if taken, is saved to the patient's profile in the system.

During the session, the patient's name, the text “NEW” until an ID is added, and location are on-screen at all times during the session. The nurse confirms the patient's information and identifies whether or not there is a duplicate account with the same information (last name, date of birth, authentication, etc.). If necessary, the patient's information is updated by the nurse from the patient's profile. If necessary, the current patient profile is changed from an incorrect profile to the correct one. If the patient's photograph is too blurry, the nurse retakes the photograph through the booth. The nurse verifies the patient and proceeds with the session.

The nurse selects “Select New Patient.” The patient list appears on the screen with the option “Add Patent” at the top. The nurse selects “Add Patient.” The nurse is prompted to assign the patient a patient ID through a dosing and inventory software linked to the booth. The patient ID is displayed to the patient on the screen.

In both of these examples, the nurse is prompted to send a dose for the patient through an Electronic Medical Record (EMR) linked to the patient's identity. The nurse views the dosing information and confirms the dose to be dispensed. The dose is generated and images are sent to the nurse before the dose is released to the patient. The nurse views the images and verifies the doses. The nurse is prompted to dispense the doses to the patient. The nurse enters a Personal Identification Number (PIN) to confirm that the dose should be dispensed. If the incorrect PIN is entered, an error message is displayed.

The patient is instructed to consume their dose in the booth. The nurse views the patient as they take their dose and confirms that they have done so. If applicable, the nurse is prompted to confirm or cancel the preparation of take-home doses. If confirmed, the doses are prepared and images of the doses are sent to the nurse for review and approval. The take-home doses are quarantined. The nurse is prompted to dispense the doses to the patient. The nurse enters a Personal Identification Number (PIN) to confirm that the dose should be dispensed. If the incorrect PIN is entered, an error message is displayed.

The nurse confirms that the patient retrieved their take-home doses before exiting the booth. If the take-home doses were not taken and the patient has left, the doses are locked down and the onsite staff is notified. The nurse then has the option to end the session. The nurse has multiple sessions at multiple locations active at once and may switch to other sessions while waiting for doses to be dispensed in one session.

In a third example, a nurse waits for a session with a patient to come into the central queue. The nurse is notified that there is a patient ready for a session and lists out the following details of the patient: location, first name, last name, patient ID, optionally the text “NEW PATIENT” in the case of a new patient, a date of birth, and a wait time. The nurse clicks on the patient's profile, views the information tied to the patient's profile, and starts the session.

In a fourth example, a nurse waits for a session with a patient to come into the central queue. A request for a session comes in. The nurse selects the requests and establishes communication with the patient. From there, the nurse uses the patient's information to search for an accompanying profile and selects it to verify the patient's identity.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for remotely approving and dispensing medications directly to a patient for opioid addiction treatment, the method comprising:
a. determining an identity of the patient, by a biometric sensing component (210);
b. establishing remote communication between the patient and a medical official, wherein the medical official is certified for treating opioid addiction;
c. transmitting a medical record corresponding to the patient to the medical official;
d. generating, by the medical official, the opioid addiction treatment;
e. generating an event record comprising the identity of the patient, the opioid addiction treatment, the medical record, and the medical official; and
f. transmitting the event record to a dosing location.

2. The method of claim 1, wherein the biometric sensing component (210) comprises a fingerprint sensor, a retina scanner, a facial recognition component, or a combination thereof.

3. The method of claim 1 further comprising providing a private booth configured to provide the remote communication between the patient and the medical official.

4. The method of claim 1, wherein the opioid addiction treatment comprises a dose of methadone, a dose of buprenorphine, or a combination thereof.

5. The method of claim 1, wherein the medical official comprises a pharmacist, a nurse, or a doctor.

6. A computing system (100) for remotely approving and dispensing medications directly to a patient for opioid addiction treatment, the system (100) comprising:
a. a communication component (110) configured to communicatively couple to a plurality of patient devices;
b. a processor (120) operatively coupled to the communication component (110), configured to execute computer-readable instructions; and
c. a memory component (130) operatively coupled to the processor (120), comprising computer-readable instructions for:
i. accepting biometric data from a patient device (200) of the plurality of patient devices;
ii. confirming an identity of the patient based on the biometric data;
iii. accessing a medical record of the patient based on the confirmed identity;
iv. establishing, by the communication component (110), communication between a medical official and the patient through the patient device (200), wherein the medical official is certified for treating opioid addiction;
v. accepting the opioid addiction treatment from the medical official;
vi. generating an event record comprising the identity of the patient, the opioid addiction treatment, the medical record, and the medical official; and
vii. transmitting the event record to a dosing location.

7. The system (100) of claim 6, wherein the biometric data comprises fingerprint data, retina data, facial data, or a combination thereof.

8. The system (100) of claim 6, wherein the plurality of patient devices comprise personal computing devices, portable computing devices, private booths, or a combination thereof.

9. The system (100) of claim 6, wherein the opioid addiction treatment comprises a dose of methadone, a dose of buprenorphine, or a combination thereof.

10. The system (100) of claim 6, wherein the medical official comprises a pharmacist, a nurse, or a doctor.

11. A computing system (1000) for remotely approving and dispensing medications directly to a patient for opioid addiction treatment, the system (1000) comprising:
a. a plurality of patient devices, each patient device (200) comprising:
i. a biometric sensing component (210) configured to accept biometric data from the patient;
ii. a first communication component (220);
iii. a first processor (230) operatively coupled to the biometric sensing component (210) and the first communication component (220), configured to execute computer-readable instructions; and
iv. a first memory component (240) operatively coupled to the first processor (230), comprising computer-readable instructions for:
A. accepting the biometric data from the biometric sensing component (210); and
B. transmitting, by the first communication component (220), the biometric data to a hub computing system (100); and
b. the hub computing system (100) comprising:
i. a second communication (110) component configured to communicatively couple to the plurality of patient devices;
ii. a second processor (120) operatively coupled to the second communication component (110), configured to execute computer-readable instructions; and
iii. a second memory component (130) operatively coupled to the second processor (120), comprising computer-readable instructions for:
A. accepting biometric data from a patient device (200) of the plurality of patient devices;
B. confirming an identity of the patient based on the biometric data;
C. accessing a medical record of the patient based on the confirmed identity;
D. establishing, by the second communication component (110), communication between a medical official and the patient through the first communication component (220) of the patient device (200), wherein the medical official is certified for treating opioid addiction;
E. accepting the opioid addiction treatment from the medical official;
F. generating an event record comprising the identity of the patient, the opioid addiction treatment, the medical record, and the medical official; and
G. transmitting the event record to a dosing location.

12. The system (1000) of claim 11, wherein the biometric sensing component (210) comprises a fingerprint sensor, a retina scanner, a facial recognition component, or a combination thereof.

13. The system (1000) of claim 11, wherein the plurality of patient devices comprise personal computing devices, portable computing devices, private booths, or a combination thereof.

14. The system (1000) of claim 11, wherein the opioid addiction treatment comprises a dose of methadone, a dose of buprenorphine, or a combination thereof.

15. The system (1000) of claim 11, wherein the medical official comprises a pharmacist, a nurse, or a doctor.
